(19) United States
(12) Patent Application Publication
(10) Pub. No.: US 2010/0160812 A1
(43) Pub. Date: Jun. 24, 2010

(54) Fine Needle Aspiration Handle Attachment

(76) Inventors: Gary JORDAN, Litchfield, NH (US); Michael ZUPKOFSKA, Rockland, MA (US)

Correspondence Address: FAY KAPLUN & MARCIN, LLP, 150 BROADWAY, SUITE 702, NEW YORK, NY, 10038, US

(21) Appl. No.: 12/546,364

(22) Filed: Aug. 24, 2009

Related U.S. Application Data
(60) Provisional application No. 61/140,775, filed on Dec. 24, 2008.

(51) Int. Cl. A61B 10/02

(52) U.S. Cl. 600/562

(57) ABSTRACT

A removable attachment for selectively connecting coaxial medical instruments comprises a first instrument having a proximal end, a distal end and a lumen extending therethrough. The first instrument comprises an arm extending from a fulcrum located on the first instrument and a second instrument movably placed within the lumen of the first instrument, the second instrument comprising a hub at a proximal end thereof. The hub comprises a distal edge to engage a proximal end of the first instrument. The arm pivots around the fulcrum and is configured to selectively engage the hub to provide a removable locking connection between the first and second instruments.

### PRIORITY CLAIM

This application claims the priority to the U.S. Provisional Application Ser. No. 61/140,775, entitled “Fine Needle Aspiration Handle Attachment” filed on Dec. 24, 2008. The specification of the above-identified application is incorporated herewith by reference.

### BACKGROUND

Biopsies may be performed with fine needle aspiration (“FNA”) devices to obtain small samples of tissue for cytology studies, endoscopy or oncology (e,g., for biopsy of the breast or liver). Biopsy needles enable the capture of histological samples from a predetermined depth within a living body and are generally controlled by mechanisms selectively attached to proximal ends thereof which remain external to the body during use. Presently available gripping handles require that an endoscope be threaded thereonto. However, as the endoscope is threaded onto the gripping handle, the entire handle and, consequently, the entire length of the FNA device is rotated winding up the FNA device which, as noted above, may be 250 cm or longer. This winding up of the FNA device creates resistance to the threading motion and, as soon as the handle is released, the FNA device begins to unwind, unthreading the endoscope from the handle.

### SUMMARY OF THE INVENTION

The present invention is directed to a removable attachment for selectively connecting coaxial medical instruments comprising a first instrument having a proximal end, a distal end and a lumen extending therethrough, the first instrument comprising an arm extending from a fulcrum located on the first instrument and a second instrument movably placed within the lumen of the first instrument, the second instrument comprising a hub at a proximal end thereof, the hub comprising a distal edge to engage a proximal end of the first instrument, wherein the arm pivots around the fulcrum and is configured to selectively engage the hub to provide a removable locking connection between the first and second instruments.

The present invention is further directed to a device for facilitating insertion of a flexible instrument into a living body comprising an elongated body extending longitudinally from a proximal end to a distal end which, when the flexible instrument is in an operative position within the body, remains outside the body accessible to a user, the body defining a lumen extending therethrough and a gripping mechanism pivotally mounted to the elongated body for movement between a gripping configuration in which an abutting surface thereof extends over a distal end of the elongated body from a radially outer edge thereof a predetermined distance toward a longitudinal axis thereof to engage a corresponding abutting surface of a flexible instrument inserted through the lumen, and an open configuration in which the abutting surface is pivoted radially beyond the outer edge of the elongated body.

### BRIEF DESCRIPTION OF THE DRAWINGS

FIG. 1 shows a first side view of a device according to a first exemplary embodiment of the present invention in a closed configuration;

FIG. 2 shows a second side view of the device of FIG. 1 in a partially open configuration;

FIG. 3 shows a third side view of the device of FIG. 1 in an open configuration;

FIG. 4 shows a second embodiment of the present invention in a closed configuration;

FIG. 5 shows a first view of a third embodiment of the present invention in an open configuration; and

FIG. 6 shows a second view of the device of FIG. 5 in a closed configuration.

### DETAILED DESCRIPTION

The present invention may be further understood with reference to the following description and the appended drawings, wherein like elements are referred to with the same reference numerals. The present invention relates to devices for performing biopsy procedures using fine-needle aspiration (“FNA”) devices. In particular, embodiments of the present invention may be employed with FNA devices for treatment of the gastrointestinal tract where a length of the FNA device exceeds 250 cm. However, it is noted that embodiments of the present invention may be applied to any FNA procedure performed at any depth in the body.

An attachment mechanism 100 according to the present invention provides an apparatus by which an endoscope or other device to be inserted into a living body to perform an FNA procedure may be secured to a gripping handle. The gripping handle may be manipulated by a physician or other user of the device to control the FNA device in situ. The attachment mechanism 100 bypasses the prior threading process by providing a lever 102 which may be selectively actuated to operate gripping arms thereof to engage the FNA device.

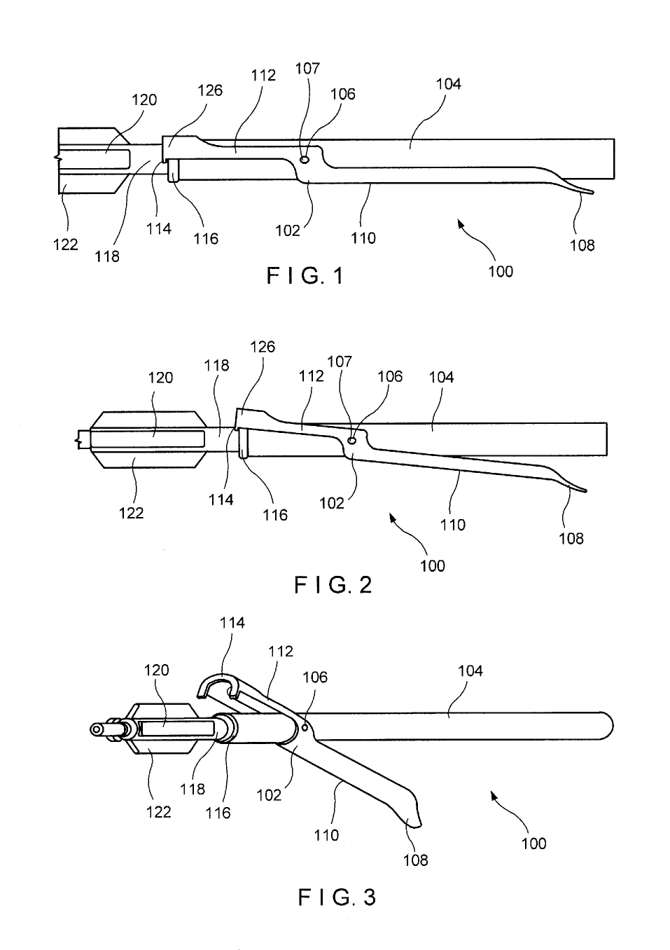

As shown in FIGS. 1-3, the attachment mechanism 100 comprises the lever 102 mounted on a distal portion of a handle body 104 which defines a lumen 101 through which an endoscope is inserted. It is noted that the use of the term distal herein refers to a direction away from a user (i.e., toward a patient on which it is being used) while the term proximal refers to a direction approaching a user of the device. The proximal portion of the endoscope and the entire attachment mechanism 100 remain external to the patient as the distal portion of the endoscope is inserted through the handle to a target site within the patient's body. Exemplary materials for the present invention may comprise any of a variety of suitable plastics, metals or combinations thereof as known to those of skill in the art so long as the materials are biocompatible and exhibit the required mechanical properties (e.g., a desired degree of rigidity, etc.). The handle body 104 and a proximal portion of an endoscope 120 inserted therethrough remain external to the body when in an operative configuration. In use, the handle body 104 provides a hand hold for gripping and manipulation of the device via which a user may apply torque and otherwise manually control the movement of the FNA device attached thereto.

The lever 102 is pivotally attached to the handle body 104 via pins 106 located on opposite lateral sides thereof which are received in corresponding bores 107 formed in the handle body 104. The pins 106 extend radially inward a predetermined distance (e.g., a distance equivalent to a depth of the bores 107). The lever 102 may then be slidably received over the handle body 104 until the pins 106 engage the bores 107, locking the lever 102 in place. The pins 106 may be substantially cylindrical and correspond in shape and size to bores 107 so that the lever 102 may pivot about an axis defined by the bores 107 when locked to the handle body 104. The pivoting motion of the lever 102 allows a user to selectively move the lever 102 between open and closed configurations, as described in greater detail below. In an alternate embodiment, the lever 102 may be pivotally attached to the handle body 104 by pins integrally formed with the lever 102 and projecting radially inward therefrom at the same spots shown for the pins 106. A portion of the lever 102 including the pins would be formed as a collar flexed radially outward and slid over the handle body 104 until the pins reach the bores 107 and are pushed thereinto by the bias of the lever 102 and maintained in this position by the bias of the lever 102. It is further noted that the pins 106 do not extend through the handle body 104 as the endoscope must be slid therethrough. The pins 106 extend only into the wall of the handle body 104 and do not penetrate the lumen 101. As would be understood by those skilled in the art, the collar (not shown) may be secured to the handle body 104 via insert molding, compression fitting, thermal bonding or another known means.

The lever 102 extends proximally and distally from the pins 106 along opposite sides of the handle body 104 (i.e., the portion of the lever 102 extending proximally from the pins 106 is on a side of a longitudinal axis of the handle body 104 opposite the portion extending distally therefrom). Specifically, a proximal portion 110 of the lever 102 extends to a tab 108 along a first longitudinal length of the handle body 104. The tab 108 facilitates grasping of the lever 102 by a user to move the lever 102 from a closed configuration shown in FIG. 1 to an open configuration shown in FIG. 3. Actuation may comprise one of application of a radially outwardly directed force to the tab 108 (e.g., by sliding a finger between the lever 102 and the handle body 104). A distal portion 112 of the lever 102 extends along a second longitudinal length of the handle body 104 to a tine 114 projecting radially inward from a distal end of the lever 102. The tine 114 according to this embodiment is formed as a part of a circle which, when in the closed configuration, is centered at the longitudinal axis of the handle body 104. A proximally facing portion of the tine 114 is tapered. In a preferred embodiment, the proximal portion 110 forms an angle of less than 180° with respect to the distal portion 112, thus permitting the lever 102 to apply a radially constrictive force on a female luer 118 of the endoscope 120 when assuming a closed configuration, as described in greater detail below.

A distal end of the handle body 104 comprises a male luer 116 comprising an opening to house the female luer 118 of the endoscope 120, as those skilled in the art will understand. The female luer 118 may be formed to fit within the male luer 116 with a fluid-tight friction fit and may be prevented from advancing into the male luer 116 beyond a predetermined distance. For example, the male luer 116 may have a depth selected to prevent the female luer 118 from being inserted thereinto beyond a predetermined depth. When in the closed configuration of FIG. 1, the tine 114 extends over and engages a luer thread (not shown) formed on the female luer 118. Engagement with at least one of the luer threads locks the female luer 118 in place against the male luer 116. Thus, when in the closed configuration, the tine 114 prevents the endoscope 120 from separating from the attachment mechanism 100. It will be appreciated by those skilled in the art that the pins 106 engage the handle body 104 with friction fit sufficient to prevent pivotal movement of the lever 102 when not moved to the open configuration by the user. FIG. 2 shows the lever 102 as it is being rotated out of engagement with the female luer 11 8 of the endoscope 120. Once the tine 114 has been moved radially out of contact with the endoscope 120, the endoscope 120 may be manually withdrawn therefrom. Arms 122 of the endoscope 120 are provided to aid in the manual manipulation of the endoscope 120.

The endoscope 120 may further be provided with a means to prevent unwanted rotation thereof once locked to the attachment mechanism 100. For example, the endoscope 120 may be provided with at least one boss (not shown) at a proximal end of the female luer 118. The tine 114 may comprise a protrusion (not shown) sized and located to engage a lateral side of the at least one boss (not shown). Specifically, when the tine 114 is in a closed configuration, the protrusion (not shown) is located adjacent to the boss (not shown) preventing rotation of the endoscope 120 in a direction approaching the protrusion. A second boss (not shown) and a second protrusion (not shown) may then be employed to prevent rotation of the endoscope 120 in the opposite second direction, as those skilled in the art will understand.

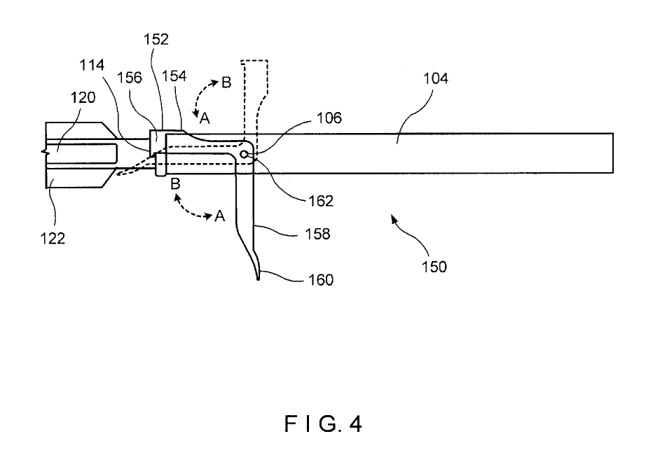

FIG. 4 depicts a locking mechanism 150 according to a second embodiment of the present invention, wherein like elements are indicated with like reference numerals. The locking mechanism 150 is formed substantially similar to the locking mechanism 100 of FIGS. 1-3 with the exception of a lever 152 formed thereon. The lever 152 is movable from an open configuration (shown in phantom) wherein a tine 114 of a distal portion 154 is radially separated from the female luer 118 of the endoscope 120 and a closed configuration wherein the proximal portion 158 is substantially perpendicular to the handle body 104 and the distal portion 154 lies flush therewith, the tine 114 lying in a contacting configuration with the female luer 118 of the endoscope 120.

Specifically, the distal portion 154 of the lever 152 is configured to be substantially perpendicular to the proximal portion 158. A joint 162 between the distal portion 154 and the proximal portion 158 is configured to receive the pins 106 on opposite lateral sides thereof. The pins 106 are received in corresponding bores 107 formed in the handle body 104 and permit rotation of the lever 152 in directions A and B. The proximal portion 158 of the lever extends along a second longitudinal length of the handle body 104 in a direction substantially opposite to a first longitudinal length housing the distal portion 154. The proximal portion 158 comprises a gripping tab 160 formed at a proximal end thereof, wherein a curvature of the proximal portion 158 and the gripping tab 160 substantially conforms to a curvature of a contacting portion of the handle body 104, as shown in phantom in FIG. 4. Furthermore, in order to permit the proximal portion 158 to lie flush against the handle body 104 in the open configuration, the proximal portion 158 may be formed with a longitudinal slot (not shown) running along a longitudinal centerline thereof, the slot defining lateral sides configured to contact the handle body 104, as those skilled in the art will understand.

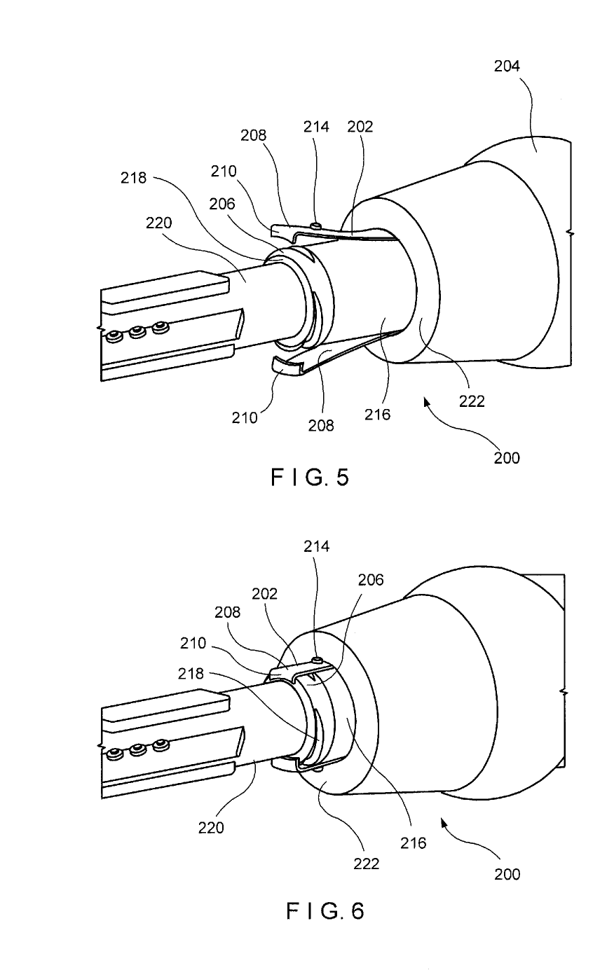

As shown in FIGS. 5-6, a locking mechanism 200 according to a third embodiment of the invention comprises a collet 202 on a distal end of a handle body 204. The collet 202 comprises two arms 208 extending distally from the handle body 204, each arm 208 further comprising an abutment 210 facing radially inward at a distal end thereof. The abutments 210 are sized and shaped to engage a shoulder of luer threads 206 of an endoscope 220 to which the handle body 204 is to be connected. As indicated in the figure, the endoscope 220 comprises one or more helical or circular luer threads 206 on a proximal end thereof.

The collet 202 is movable between a retracted position housed within a handle luer 216, and an actuated position in which the collet 202 is extended distally from distal end of the handle body 204. Proximal and distal movement of the handle body 204 relative to the collet 202 may be performed via actuation of a lever (not shown) on a proximal portion of the handle body 204, as those skilled in the art will understand. For example, the lever may be held in position to maintain the position of the collet 202 substantially constant as the handle body 204 is moved proximally to move the collet 202 out of the handle body 204. The arms 208 of the collet 202 are biased toward a bent configuration in which the distal ends thereof are spread radially from the one another and are radially separated from the surface of the handle luer 216. Thus, as the collet 202 is moved distally to the actuated position, the handle body 204 no longer constrains the arms 208 and they splay radially outward due to the bias. Then, the lever may be held in position as the handle body 204 is moved distally over the arms 208 drawing them radially inward to engage the luer 218 of the endoscope 220. Movement of the handle body 204 distally over the collet 202 can proceed until a post 214 engages a distal face 222 of the handle body 204. This prevents the collet 202 from being drawn into the handle body 204 beyond a predetermined distance. In this manner, both a length of the arms 208 and the abutments 210 apply a retaining force on the endoscope 220 to lock a configuration thereof. The abutments 210 extend radially inward by a distance selected to engage the luer 218 in a manner sufficient to maintain the endoscope 204 connected to the handle body 204 until manually disengaged by the user. As would be understood by those skilled in the art, the user may disengage the arms 208 from the endoscope by holding the lever in position to maintain the position of the collet 202 substantially constant as the handle body 204 is moved proximally, extending the arms 208 from the handle body 204 and returning the collet 202 to the open position.

It will be apparent to those skilled in the art that various modifications may be made in the present invention, without departing from the spirit or scope of the invention. Thus, it is intended that the present invention cover modifications and variations of this invention provided that they come within the scope of the appended claims and their equivalents.

What is claimed is:

1. A removable attachment for selectively connecting coaxial medical instruments comprising:
a first instrument having a proximal end, a distal end and a lumen extending therethrough, the first instrument comprising an arm extending from a fulcrum located on the first instrument; and
a second instrument movably placed within the lumen of the first instrument, the second instrument comprising a hub at a proximal end thereof, the hub comprising a distal edge to engage a proximal end of the first instrument, wherein the arm pivots around the fulcrum and is configured to selectively engage the hub to provide a removable locking connection between the first and second instruments.

2. The removable attachment of claim 1, wherein the fulcrum comprises a pin formed on the first instrument, the fulcrum defining an axis about which the arm pivots

3. The removable attachment of claim 3, wherein the pin is received in a bore formed in a wall of the first instrument, an end of the bore preventing entry of the pin into the lumen.

4. The removable attachment of claim 1, wherein the arm comprises a proximal portion on a first side of the fulcrum and a distal portion on a second side of the fulcrum diametrically opposed to the first side.

5. The removable attachment of claim 4, wherein a proximal end of the proximal portion is formed as a handle which, when in a gripping configuration, angles away from an outer surface of the first instrument.

6. The removable attachment of claim 5, wherein the arm is formed so that, when in the gripping configuration, the distal portion and the proximal portion distal of the handle lie flush against the outer surface of the first instrument.

7. The removable attachment of claim 5, wherein the proximal portion is configured to lie in a plane forming an angle of less than 180° with a plane in which the distal potion lies.

8. The removable attachment of claim 4, wherein the proximal portion is perpendicular to the distal portion.

9. The removable attachment of claim 1, wherein the arm is partially retracted into the first instrument body in a gripping configuration.

10. A device for facilitating insertion of a flexible instrument into a living body, comprising:
an elongated body extending longitudinally from a proximal end to a distal end, the body defining a lumen extending therethrough; and
a gripping mechanism extending from a proximal end comprising a handle to a distal end comprising an abutting surface, the gripping mechanism pivotally mounted to the elongated body for movement between a gripping configuration in which the abutting surface engages a corresponding abutting surface of the flexible instrument inserted through the lumen and an open configuration in which the abutting surface is radially separated from the elongated body.

11. The device of claim 10, wherein the gripping mechanism includes an arm mounted to the elongated body via a pin forming an axis about which the arm pivots, the arm including a proximal portion on a first side of the elongated body and a distal portion on a second side of the elongated body diametrically opposed to the first side.

12. The device of claim 11, wherein the pin and the arm are integrally formed.

13. The device of claim 11, wherein the pin is received in a bore formed in a wall of the elongated body, an end of the bore preventing entry of the pin into the lumen of the elongated body.

14. The device of claim 11, wherein the arm is formed so that, when in the gripping configuration, the distal portion of the arm and the proximal portion of the arm lie flush against the outer surface of the elongated body.

15. A device for facilitating insertion of a flexible instrument in a living body, comprising:
a body portion extending from a proximal end to a distal end;
a gripping mechanism comprising a first arm movable from an open configuration in which the first arm extends distally out of the body portion in a radially expanded configuration to a gripping configuration in which the first arm is partially retracted into the body portion; and
a first abutting surface formed on a distal end of the gripping mechanism and configured to engage a proximal portion of a flexible instrument received within the body portion.

16. The device of claim 15, further comprising a second arm positioned to be diametrically opposed to the first arm, the second arm further comprising a second abutting surface formed on a distal end thereof configured to engage a proximal portion of the flexible instrument.

17. The device of claim 16, wherein the second arm is perpendicular to the first arm.

18. The device of claim 15, wherein the first arm further comprises an abutment formed thereon to prevent retraction of the first arm into the body portion therepast.

19. The device of claim 15, wherein the gripping mechanism is actuated by a handle located on a proximal end of the body portion located external to the body in an operative configuration.
